(19) United States
(12) Patent Application Publication
(10) Pub. No.: US 2022/0273694 A1
(43) Pub. Date: Sep. 1, 2022

(54) COMPOSITIONS AND METHODS FOR DECREASING INTRAOCULAR PRESSURE

(71) Applicant: Duke University, Durham, NC (US)

(72) Inventors: Pedro GONZALEZ, Durham, NC (US); Coralia C. LUNA, Durham, NC (US)

(73) Assignee: Duke University, Durham, NC (US)

(21) Appl. No.: 17/633,024

(22) PCT Filed: Aug. 7, 2020
(86) PCT No.: PCT/US2020/045463

§ 371 Date: Feb. 4, 2022

Related U.S. Application Data
(60) Provisional application No. 62/883,768, filed on Aug. 7, 2019.

(51) Int. Cl. A61K 31/7105; A61K 31/711; A61K 45/06; A61P 27/02; C12N 15/113

(52) U.S. Cl. CPC A61K 31/7105; A61K 31/711; A61K 45/06; A61P 27/02; C12N 15/113; C12N 2310/113; C12N 2310/141; C12N 2320/32; C12N 2710/10343; C12N 2740/16043; C12N 2750/14143

(57) ABSTRACT

Provided herein are methods and compositions for decreasing intraocular pressure in a subject. Also provided are methods and compositions for treating or preventing glaucoma.

### PRIOR RELATED APPLICATION

This application claims the benefit of and priority to U.S. Provisional Application No. 62/883,768 filed on Aug. 7, 2019, which is hereby incorporated by reference in its entirety.

### STATEMENT REGARDING FEDERALLY FUNDED RESEARCH

This invention was made with government support under Grant No. R21EY029400 awarded by the National Institutes of Health/National Eye Institute. The government has certain rights in the invention.

### FIELD

This disclosure describes compositions and methods for decreasing intraocular pressure.

### Reference to a Sequence Listing Submitted as a Text File Via EFS-Web

The official copy of the sequence listing is submitted electronically via EFS-Web as an ASCII formatted sequence listing with a file named 1202756 seqlist.txt, created on Aug. 6, 2020, and having a size of 11 kb and is filed concurrently with the specification. The sequence listing contained in this ASCII formatted document is part of the specification and is herein incorporated by reference in its entirety.

### BACKGROUND

Glaucoma is a leading cause of irreversible blindness in the world. It is estimated that more than three million Americans and over 60 million people worldwide have glaucoma. Ocular hypertension, or increased intraocular pressure is a main risk factor for glaucoma. Lowering intraocular pressure is currently the best approach to treat or prevent glaucoma. Unfortunately, the efficacy of current methods for lowering intraocular pressure is hampered by patient non-compliance. Composition and methods for lowering intraocular pressure, that are largely independent from patient compliance, are necessary.

### SUMMARY

Provided herein are methods for decreasing intraocular pressure (IOP) in a subject. The methods comprise increasing expression of one or more microRNAs (miRNAs) in the eye of a subject in need thereof, wherein the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

Also provided are methods for treating glaucoma in a subject. The methods comprise increasing expression of one or more microRNAs (miRNAs) in the eye of a subject in need thereof, wherein the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

In some embodiments, expression of the one or more miRNAs is increased by administering to the eye of a subject in need thereof one or more miRNAs having at least 95% identity to a nucleic acid sequence selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

In some embodiments, one or more DNA sequences that encode the one or more miRNAs are administered to the eye of the subject.

In some embodiments, expression of the one or more miRNAs is increased in one or more cells of the outflow pathway of the subject. In some embodiments, the one or more cells are selected from the group consisting of a trabecular meshwork cell, a Schlemm's canal cell, and a juxtacanalicular cell.

In some embodiments, the one or more miRNAs or the one or more DNA sequences encoding the one or more miRNAs is in a viral vector. In some embodiments, the viral vector is an adeno-associated viral vector or a lentiviral vector. In some embodiments, the one or more miRNAs are operatively linked to a promoter. In some embodiments, the promoter comprises a cytomegalovirus (CMV) enhancer fused to the chicken beta-actin promoter (CAG promoter) sequence. In some embodiments, the promoter comprises an eye tissue-specific promoter.

In some embodiments, the one or more miRNAs or the one or more DNA sequences encoding the one or more miRNAs is administered to the eye of the subject via non-viral delivery. In some embodiments, non-viral delivery comprises nanoparticle delivery.

In some embodiments, the method further comprises administration of one or more agents selected from the group consisting of a prostaglandin analog, a beta blocker, an alpha-adrenergic agonist, a carbonic anhydrase inhibitor, and a cholinergic agent. In some embodiments, the subject has had eye surgery to reduce IOP. In some embodiments, the method further comprises conducting eye surgery on the subject to reduce IOP.

Also provided herein is a vector comprising a nucleic acid sequence encoding a miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5). In some embodiments, the vector is a viral vector. In some embodiments, the vector is a DNA vector or RNA vector. In some embodiments, the vector is an adeno-associated viral vector or lentiviral vector. Also provided is a cell comprising any of the vectors described herein.

Also provided is a nanoparticle comprising a miRNA or a DNA sequence encoding an miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

Also provided is a pharmaceutical composition comprising any of the vectors or nanoparticles described herein.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows that effects of miR-146a expression on IOP of ten Brown Norway rats injected in one eye with 15 μL of 7.5×107 pfus of an adenoviral vector expressing miR-146a, under the control of the CAG promoter system according to certain embodiments of this disclosure. IOPs were monitored using a Tonolab rebound tonometer for more than two months. Injected eyes (miR-146a) showed a decrease in TOP that was statistically significant (p<0.05) for day 4 and after day 14, until the end of the experiments at day 63.

FIG. 2 shows that injection of a control virus with the CAG promoter system and no insert had no in statistically significant impact in TOP according to certain embodiments of this disclosure. (Average difference=0.35897619 mmHg ±0.84573066 n=6).

FIG. 3 shows the effects of miR-146a expression on TOP of three Brown Norway rats injected in one eye with 25 μL of 5.6×108 pfus/mL lentiviral vector expressing miR-146a under control of the CAG promoter according to certain embodiments of this disclosure. The injected eyes (bottom line) showed a consistent decrease in TOP with no obvious abnormalities or signs of inflammation in the injected eyes when observed under a stereomicroscope.

FIG. 4 shows the effects of lentiviral administration of miR-146a on TOP according to certain embodiments of this disclosure. Six Brown Norway rats were injected in one eye with 15 μL of a lentivirus expressing miR-146a at 1×109 pfu/mL. The injected eyes showed a decrease in TOP that was statistically significant (p<0.05) for more than eight months after injection.

FIG. 5 shows that average difference in TOP was 4.452±2.928 (n=6) for the six Brown Norway rats described in FIG. 5 that were injected in one eye with 15 μL of a lentivirus expressing miR-146a at 1×109 pfu/mL according to certain embodiments of this disclosure.

FIG. 6A shows the effects of lentiviral administration of miR-146a on visual function according to certain embodiments of this disclosure. Six rats injected with 15 μL of a lentivirus expressing miR-146 at 1×109 pfu/mL were evaluated for potential alterations in visual function one-and-a-half months and seven months after injection. No statistical difference in visual acuity was observed between injected and non-injected control eyes.

FIG. 6B shows representative images of 6 rat eyes injected with lenti-CAG-miR-146a 8 months after injection according to certain embodiments of this disclosure.

FIG. 7 shows that expression of inflammatory genes in the anterior chamber of rat eyes transduced with miR-146a did not show significant differences compared to controls eyes according to certain embodiments of this disclosure. miR-146a showed significant up-regulation in rat eyes transduced with lenti-miR-146a, at 9 months after injection (N=3).

FIG. 8 shows that semi-thin sections of rat eyes, angle transduced with lenti-miR-146a or contralateral control eyes, did not exhibit any noticeable differences among them, nine months after injection, according to certain embodiments of this disclosure. Panels A, B, and C are representative images of miR-146a transduced eyes. Panels D, E and F are representative images of contralateral eyes (N=3). SC=Schlemm's canal; TM=trabecular meshwork.

FIG. 9 shows the effects of miR-146a on HTM cells at basal level and under cyclic mechanical stress according to certain embodiments of this disclosure. Two primary HTM cells (L3 and 1788) were transduced with miR-146a mimic (146M), which downregulated the expression of most of the analyzed genes in one or both primary cells, compared to negative control (scramble) transduced cells. When mechanical force was applied, transduced with miR 146a (146M Str) reduced or cancelled the increase in expression induced by the stretching for most of the analyzed genes. Gene expression of cells transduced with 146M (no CMS) (left column for each gene), 146M-Str (CMS) (middle column for each gene) and scramble-Str (CMS) (right column for each gene) are represented as a percentage of cells transduced with negative control (scramble, no CMS) (horizontal line at 100).

FIG. 10 shows the effects of an miR-146 inhibitor on HTM cells at basal level and under cyclic mechanical stress according to certain embodiments of this disclosure. Two primary HTM cells (L3 and 1788) transduced with miR-146 inhibitor (146I) upregulated the expression of many analyzed genes in one or both primary cells, compared to negative control (scramble) transduced cells. When mechanical force was applied (CMS), cells transduced with miR 146I (146I Str) increased the expression of some genes compared to scramble stretching. Gene expression of cells transduced with 146I (no CMS) (left column for each gene), 146I-Str (CMS) (right column for each gene) and scramble-Str (CMS) (middle column for each gene) are represented as a percentage of cells transduce with negative control (scramble, no CMS) (horizontal line at 100).

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Ocular hypertension occurs when pressure inside the eye (IOP) is higher than normal. With ocular hypertension, the front of the eye does not drain fluid properly causing eye pressure to build up. Higher than normal eye pressure can cause glaucoma. To date, gene therapy to decrease IOP has been largely unsuccessful, or limited to certain conditions. Further, these treatments often resulted in adverse effects.

For example, targeting genes of the prostaglandin pathway resulted in weak decreases in TOP and caused uveitis (Barraza et al. “Prostaglandin pathway gene therapy for sustained reduction of intraocular pressure. Mol Ther. 2010 March; 18(3):491-501; Lee et al. Prospects for lentiviral vector mediated prostaglandin F synthase gene delivery in monkey eyes in vivo. Curr Eye Res. 2014 September; 39(9):859-70). AAV delivery of tissue plasminogen activator in the trabecular meshwork attenuated the increase of IOP caused by steroids, but did not result in a significant decrease of IOP in normal eyes (Kumar et al. Tissue plasminogen activator in trabecular meshwork attenuates steroid induced outflow resistance in mice. PLoS One. 2013 Aug. 19; 8 (8):e72447). Similarly, AAV mediated delivery of inducible MMP1 prevented the IOP increase produced by corticosteroid instillation in a sheep model, but did not decrease IOP levels in eyes that were not treated with steroids (Spiga et al. Development of a gene therapy virus with a glucocorticoid-inducible MMP1 for the treatment of steroid glaucoma. Invest Ophthalmol Vis Sci. 2010 June; 51(6):3029-41). Delivery of Clostridium botulinum exoenzyme C3 transferase (C3), a toxin known to disrupt the actin cytoskeleton, was delivered to the anterior chamber of rodent and non-human primates using both lentiviral and adeno associated vectors. The effects on IOP were not durable using lentiviral vectors in both rodents and monkeys. Delivery with AAV vectors resulted in severe corneal edema and weak IOP response. (Slauson et al. Viral Vector Effects on Exoenzyme C3 Transferase-Mediated Actin Disruption and on OutflowFacility. Invest Ophthalmol Vis Sci. 2015 April; 56(4):2431-8; Tan et al. Effects of Lentivirus-Mediated C3 Expression on Trabecular Meshwork Cells and Intraocular Pressure. Invest Ophthalmol Vis Sci. 2018 Oct. 1; 59(12):4937-4944).

Until the present invention, successful gene therapy to elicit a long-term decrease in IOP, accompanied by minimal to no side effects, had not been achieved.

### I. Methods

Provided herein are methods for decreasing intraocular pressure (IOP) in a subject. The methods comprise increasing expression of one or more miRNAs in the eye of a subject. In some methods, the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

Methods for treating or preventing glaucoma in a subject are also provided. The methods comprise increasing expression of one or more miRNAs in the eye of a subject. In some methods, the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

As used herein, the term “intraocular pressure” (IOP) refers to eye pressure. IOP is measured in millimeters of mercury (mm Hg). Normal eye pressure ranges from about 10-21 mm Hg, and eye pressure greater than 22 mm Hg is considered higher than normal. When IOP is higher than normal, i.e., above 21 mm Hg, and the subject does not show signs of glaucoma, the subject may have ocular hypertension, and could be at risk for developing glaucoma, also referred to as a glaucoma suspect. The term glaucoma suspect is also used to describe those who have other findings that could potentially, now or in the future, indicate glaucoma. For example, a suspicious optic nerve, or even a strong family history of glaucoma, could put someone in the category of a glaucoma suspect.

Glaucoma is a group of eye conditions that damage the optic nerve, usually due to abnormally high pressure in the eye. Optic nerve damage cannot be reversed. The peripheral (side) vision is usually affected first. The changes in vision may be so gradual that they are not noticed until significant vision loss has already occurred. In time, if the glaucoma is not treated, central vision will also be decreased and then lost.

The methods provided herein can be used to decrease IOP in a subject, regardless of whether the subject has or is at risk of developing glaucoma. The methods provided herein can also be used to decrease IOP in a subject that has or is at risk of developing glaucoma. A decrease in IOP can be a decrease of at least about 5%, 10%, 20% or greater as compared to the IOP of the subject prior to increasing expression of one or more miRNAs in the subject, or a control value. In some methods, IOP is decreased such that the subject has a normal IOP, i.e., an IOP that is in the range of about 10-21 mm Hg. The decrease in IOP can last for at least about a week, two weeks, three weeks, a month, two months, three months, four months, five months, six months, seven months, eight months, nine months, ten months, eleven months, a year or greater. In some instances, the decrease in IOP lasts for several years up to the remaining life time of the patient.

In some methods, the measured IOP value in a patient can be compared with a control value in populations who are free of symptoms of glaucoma, for example, prophylactically treated patients who are free of symptoms of glaucoma, or populations of therapeutically treated patients who show mitigation and/or amelioration of symptoms of glaucoma. If the measured level of TOP is similar to or less than a control value, the treatment is considered successful. If the measured level of IOP in the patient is significantly above the control value, additional administration of the treatment at the same or a different dosage may be warranted.

In some embodiments, expression of the one or more miRNAs is increased by administering to the eye of a subject in need thereof one or more miRNAs having at least 95% identity to a nucleic acid sequence selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5) as set forth in Table 2.

In some embodiments, one or more DNA sequences that encode the one or more miRNAs are administered to one or both eyes of the subject. In some embodiments, a DNA sequence that encodes a precursor miRNA sequence comprising one or more mature miRNAs is administered to the eye of the subject. In some embodiments, expression of the one or more miRNAs is increased in one or more cells of the outflow pathway of the subject. In some embodiments, the one or more cells of the outflow pathway are selected from the group consisting of a trabecular meshwork cell, a Schlemm's canal cell, and a juxtacanalicular cell.

In some embodiments, the DNA sequence encodes a miR-146a precursor having at least 95% identity to SEQ ID NO: 4. In some embodiments, the DNA encoding a human miR-146a precursor (for example, SEQ ID NO: 4) encodes one or more mature human miR-146a miRNAs, for example, human miR-146a-5p (SEQ ID NO: 1) and human miR-146a-3p (SEQ ID NO: 2). In some embodiments, the DNA encodes a miR-146a stem loop structure, for example, SEQ ID NO: 6, that comprises SEQ ID NO: 1 and SEQ ID NO: 2. The stem loop structure of SEQ ID NO: 6 is shown below, with base pairing between SEQ ID NO: 1 and SEQ ID NO: 2, shown in bold.

```
      c -----u u uu c u g uc
5′ cgaug guaucc cagcu gagaacugaauu ca ggguu ug a
        ||||| |||||| ||||| |||||||||||| || ||||| || g
3′ gcuac uauagg gucga uucuugacuuaa gu uccag ac u
      u ugucuc - -c a c - ug
```

In some embodiments, the DNA sequence encodes a miR-146b precursor having at least 95% identity to SEQ ID NO: 5. In some embodiments, the DNA encoding a human miR-146b precursor (for example, SEQ ID NO: 2) encodes one or more mature human miR-146b miRNAs, for example, human miR-146b-5p (SEQ ID NO: 3) and human miR-146b-3p (SEQ ID NO: 4). In some embodiments, the DNA encodes a miR-146b stem loop structure, for example, SEQ ID NO: 7 that comprises SEQ ID NO: 3 and SEQ ID NO: 4. The stem loop structure of SEQ ID NO: 7 is shown below, with base pairing between SEQ ID NO: 3 and SEQ ID NO: 4, shown in bold.

```
          u g au cu ga u
5′ cc ggcacu agaacuga uccauagg gu gc c
      || |||||| |||||||| |||||||| || ||
3′ gg ccgugg ucuugacu aggugucc ua cg u
          c - -c cg -a a
```

In any of the methods or compositions provided herein, an miRNA can comprise, consist essentially of, or consist of a sequence having at least 95% identity to SEQ ID NO: 1, SEQ ID NO: 2, SEQ ID NO: 3, SEQ ID NO: 4, SEQ ID NO: 5 or SEQ ID NO: 6. In some embodiments, an miRNA comprises, consists essentially of, or consists of SEQ ID NO: 1, SEQ ID NO: 2, SEQ ID NO: 3, SEQ ID NO: 4, SEQ ID NO: 5 or SEQ ID NO: 6.

In the methods provided herein, expression of the one or more miRNAs, in one or both eyes of the subject, can be increased by at least 10-fold, 25-fold, 50-fold, 100-fold, 500-fold, 1000-fold, 2000-fold, 3000-fold, 4000-fold, 5000-fold or greater as compared to a control level or amount of the one or more miRNAs. A control level can be, for example, the level of the one or more miRNAs in one or both eyes of the subject, prior to administration of one or more miRNAs or a DNA sequence encoding the one or more miRNAs.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding DNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

As used throughout, miRNA can be a precursor miRNA or a mature miRNA. Such miRNAs include naturally occurring molecules as well as chemically modified miRNAs, such as miRNA agomirs. Mature miRNAs (usually about 17-24 nucleotides in length) are derived from longer precursor or pre-miRNA transcripts. See, for example, “MicroRNAs: Processing, Maturation, Target Recognition and Regulatory Functions,” Mol. Cell Pharmacol. 3(3): 83-92 (2011). An miRNA is small non-coding RNA that affects the stability or translation of mRNAs, for example, by cleaving mRNA, destabilizing mRNA through shortening of its polyA tail or causing less efficient translation of mRNA into proteins.

Unless otherwise indicated, a particular nucleic acid sequence also implicitly encompasses modified variants thereof, alleles, orthologs, SNPs, and complementary sequences as well as the sequence explicitly indicated.

The term “identity” or “substantial identity”, as used in the context of a polynucleotide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, about 20 to 50, about 20 to 100, about 50 to about 200 or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

In some embodiments, the one or more miRNAs or the one or more DNA sequences encoding the one or more miRNAs is in a non-viral (e.g., a plasmid or naked DNA) or a viral vector. In some embodiments, the vector is a viral vector. Examples of viral vectors include, but are not limited to an adeno-associated virus (AAV) vector, a retroviral vector, a herpes simplex virus, a lentiviral vector, or an adenoviral vector.

In some embodiments, the viral vector is an AAV vector comprising a 5′ inverted terminal repeat and a 3′ inverted terminal repeat. In some embodiments, the AAV vector can be a single-stranded AAV vector or a self-complementary AAV vector. Examples of AAV vectors include AAV1, AAV2, AAV3, AAV4, AAV5, AAV6, AAV7, AAV8, AAV9, AAV10, AAV11 and AAV12. In some embodiments, the viral vector is an AAV vector with a capsid protein of an AAV selected from the group consisting of AAV1, AAV2, AAV3, AAV4, AAV5, AAV6, AAV7AAV8, AAV9, AAV10, AAV11 and AAV12.

In some embodiments, the viral vector is a lentiviral vector. See, for example, Takahashi “Delivery of genes to the eye using lentiviral vectors,” Methods Mol. Biol. 246: 439-49 (2004); and Balaggan and Ali “Ocular gene delivery using lentiviral vectors,” Gene therapy 19(2): 145-153 (2011). The lentiviral genome consists of single-stranded RNA that is reverse-transcribed into DNA and then integrated into the host cell genome. Lentiviruses can infect both dividing and non-dividing cells, making them attractive tools for gene therapy.

In some embodiments, the one or more miRNAs are operatively linked to a promoter. In some embodiments, the promoter is a constitutive promoter, for example, a cytomegalovirus (CMV) promoter, a human elongation factor-1 alpha (EF1 α) promoter, a CMB enhancer fused to the chicken beta-actin promoter (CAG promoter), a phospholycerate kinase (PGK) promoter, or a U6 promoter. In some embodiments the CMV promoter comprises a sequence that has at least 95% identity to SEQ ID NO: 8, as set forth below.

(SEQ ID NO: 8)
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGAGTATTTACGGTAAACTGCCCA
CTTGGCAGTACATCAAGTGTATCATATGCCAAGTACGCCCCCTATTGACG
TCAATGACGGTAAATGGCCCGCCTGGCATTATGCCCAGTACATGACCTTA
TGGGACTTTCCTACTTGGCAGTACATCTACGTATTAGTCATCGCTATTAC
CATGGTGATGCGGTTTTGGCAGTACATCAATGGGCGTGGATAGCGGTTTG
ACTCACGGGGATTTCCAAGTCTCCACCCCATTGACGTCAATGGGAGTTTG
TTTTGGCACCAAAATCAACGGGACTTTCCAAAATGTCGTAACAACTCCGC
CCCATTGACGCAAATGGGCGGTAGGCGTGTACGGTGGGAGGTCTATATAA
GCAGAGCT.

In some embodiments the CAG promoter comprises a sequence that has at least 95% identity to SEQ ID NO: 9, as set forth below.

(SEQ ID NO: 9)
GCGTTACATAACTTACGGTAAATGGCCCGCCTGGCTGACCGCCCAACGAC
CCCCGCCCATTGACGTCAATAATGACGTATGTTCCCATAGTAACGCCAAT
AGGGACTTTCCATTGACGTCAATGGGTGGAGTATTTACGGTAAACTGCCC
ACTTGGCAGTACATCAAGTGTATCATATGCCAAGTACGCCCCCTATTGAC
GTCAATGACGGTAAATGGCCCGCCTGGCATTATGCCCAGTACATGACCTT
ATGGGACTTTCCTACTTGGCAGTACATCTACGTATTAGTCATCGCTATTA
CCATGGTCGAGGTGAGCCCCACGTTCTGCTTCACTCTCCCCATCTCCCCC
CCCTCCCCACCCCCAATTTTGTATTTATTTATTTTTTAATTATTTTGTGC
AGCGATGGGGGCGGGGGGGGGGGGGGGGCGCGCGCCAGGCGGGGCGGGGC
GGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCG
GCGGCCCTATAAAAAGCGAAGCGCGCGGCGGGCG

Inducible promoters such as the tetracycline inducible promoter or a glucocorticoid inducible promoter can also be used. The nucleic acids described herein can also be under the control of a tissue-specific promoter to promote expression of the nucleic acid in specific cells, tissues, or organs. In some embodiments, the promoter comprises an eye tissue-specific promoter. See, for example, Liton et al. “Specific Targeting of Gene Expression to a Subset of Human Trabecular Meshwork Cells Using the Chitinase 3-Like 1 promoter,” Investigative Ophthalmology & Visual Science, 46(1): 183-190 (2005).

Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

In some embodiments, the one or more miRNAs or the one or more DNA sequences encoding the one or more miRNAs is administered to the eye of the subject via non-viral delivery. Non-viral delivery methods include but are not limited to naked DNA delivery, naked miRNA delivery, inorganic material-based delivery systems, lipid-based nanocarriers, polymeric vectors, dendrimer-based vectors, cell-derived membrane vesicles and 3D scaffold based delivery systems. In some embodiments, non-viral delivery comprises nanoparticle delivery. Examples of nanoparticles that can be used in the methods and compositions described herein include, gold nanoparticles, silica nanoparticles, polyethyleneglycol/polyethyleneimine particles, or lipid nanooparticles can be used. See, for example, Lee et al. “MicroRNA delivery through nanoparticles,” Journal of Controlled Release, 313(10): 80-95 (2019). Eye drops can also be used to deliver the one or more miRNAs to the eye of the subject. See, for example, Martinez et al. “In vitro and in vivo efficacy of SYL040012, a novel siRNA compound for treatment of glaucoma,” Mol. Ther. 22(1): 81-91 (2014).

In some embodiments, the one or more miRNAs or one or more DNA sequences encoding the one or more miRNAs primarily transduce the trabecular meshwork of the eye, and are not transduced to any great extent into the cornea. The trabecular meshwork is an area of tissue located around the base of the cornea, near the ciliary body, and is responsible for draining the aqueous humor from the eye via the anterior chamber (the chamber on the front of the eye covered by the cornea). As used herein, the term “primarily” means that at least 90%, 95%, or 99% of the nucleic acids transduce the trabecular meshwork of the eye, and less than about 10%, 5% or 1% of the nucleic acids transduce corneal cells, for example, corneal epithelial cells. In some embodiments, a vector that specifically targets trabecular meshwork cells is used. In some embodiments, by primarily transducing the trabecular meshwork of the eye, adverse effects to the cornea, such as neovascularization, opacification and edema can be reduced. Cataracts, conjunctival hyperemia, and aqueous flare can also be reduced. Adverse changes in anterior chamber cells and/or vitreal cavity cells can also be reduced.

In some embodiments, a vector comprising one or more miRNAs or one or more DNA sequence encoding the one or more miRNAs can also comprise an antisense molecule, under the control of an inducible promoter, wherein the antisense molecule targets the promoter driving expression of the one or more miRNAs. Expression of this antisense can be induced, if expression of the one or more miRNAs results in a detrimental decrease in IOP, or if unwanted expression of the one or more miRNAs occurs, for example, in corneal epithelial cells. See for example, Fatal and Bochot “Ocular delivery of nucleic acids: antisense oligonucleotides, aptamers and siRNA,” Adv. Drug Deliv. Res. 58(11): 1203-23 (2006).

In some embodiments, the method further comprises administration of one or more agents selected from the group consisting of a prostaglandin analog, a beta blocker, an alpha-adrenergic agonist, a carbonic anhydrase inhibitor, and a cholinergic agent.

In some embodiments, the one or more agents are administered as an ophthalmic solution comprising a prostaglandin F2a analogue (latanoprost (Xalatan) 50 μg/ml); a Rho kinase inhibitor (for example, 0.2% Rhopressa® (netarsudil)); a beta-blocker, such as 0.5% timolol; or a carbonic anhydrase inhibitor (for example, 1% brinzolamide ophthalmic suspension). In any of the methods described herein, the one or more agents can be administered prior to, concurrently or after increasing expression of the one or more miRNAs in the eye of the subject.

In some embodiments, the subject has had eye surgery, for example, glaucoma surgery, to reduce IOP. In some embodiments, the method further comprises conducting eye surgery on the subject to reduce IOP. Glaucoma surgeries include microscopic incisional trabeculectomy, tube shunt implantation, cytophotocoagulation and minimally invasive glaucoma surgery, to name a few.

As used throughout, by subject is meant an individual. The subject can be an adult subject or a pediatric subject. Pediatric subjects include subjects ranging in age from birth to eighteen years of age. Preferably, the subject is an animal, for example, a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used herein the terms “treatment”, “treat”, or “treating” refers to a method of reducing one or more of the effects of the disease or one or more symptoms of the disease, for example, glaucoma, in the subject. Thus, in the disclosed methods, treatment can refer to a 10%, 20%, 30%, 40%, 50%, 60%, 70%, 80%, 90%, or 100% reduction in the severity of glaucoma. For example, a method for treating glaucoma is considered to be a treatment if there is a 10% reduction in one or more symptoms of glaucoma in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease or symptoms of the disease.

As utilized herein, by “prevent”, “preventing”, or “prevention” is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of glaucoma or one or more symptoms of glaucoma in a subject susceptible to glaucoma as compared to control subjects susceptible to glaucoma that did not receive a composition described herein. The disclosed method is also considered to be a prevention if there is a reduction or delay in onset, incidence, severity, or recurrence of glaucoma or one or more symptoms of glaucoma in a subject susceptible to glaucoma after receiving a composition described herein, as compared to the subject's progression prior to receiving treatment. Thus, the reduction or delay in onset, incidence, severity, or recurrence of glaucoma can be about a 10, 20, 30, 40, 50, 60, 70, 80, 90, 100%, or any amount of reduction in between.

When a disease, or a symptom thereof, is being treated, administration of any of the compositions described herein occurs after onset of the disease or symptoms thereof. In some cases, administration occurs after diagnosis of the disease. When a disease, or symptoms thereof, are being prevented, administration typically occurs before the onset of the disease or symptoms thereof. In some cases, administration occurs when a subject is diagnosed as being at risk for the disease.

As used herein, administer or administration refers to the act of introducing, injecting or otherwise physically delivering a substance as it exists outside the body (e.g., one or more miRNAs or a DNA sequence encoding the one or more miRNAs) into a subject, such as by ocular delivery. Ocular delivery includes, but is not limited to, intraocular injection, eye drops, ointments, drug/agent-impregnated contact lenses, mechanical pump, and membrane release systems. In some embodiments, any of the compositions described herein is injected into the anterior chamber of the eye. In some embodiments, a vector, for example, a lentiviral vector or an adeno-associated vector, comprising one or more miRNAs or DNA sequences encoding one or more miRNAs described herein, is intraocularly injected into the anterior chamber of the eye.

In some embodiments, a pharmaceutical composition comprising any of the compositions described herein is administered to the subject. In some embodiments, the pharmaceutical compositions will comprise sufficient amounts of one or more miRNAs or DNA sequences encoding one or more miRNAs described herein, to produce a therapeutically effective amount of the nucleic acid of interest.

As used herein, the term therapeutically effective amount or effective amount refers to an amount of a composition described herein, that, when administered to a subject, is effective to decrease TOP or treat glaucoma either by one dose or over the course of multiple doses. A suitable dose can depend on a variety of factors including the particular miRNA used and whether it is used concomitantly with other therapeutic agents. Other factors affecting the dose administered to the subject include, e.g., the type or severity of the disease. For example, a subject having ocular hypertension with no signs of glaucoma may require administration of a different dosage than a subject with glaucoma.

The effective amount of the compositions described herein can be determined by one of ordinary skill in the art. One of skill in the art will appreciate that an effective amount of a viral vector, for example, a lentiviral vector or an adeno-associated vector, can be empirically determined. An effective amount of any of the viral vectors described herein will vary and can be determined by one of skill in the art through experimentation and/or clinical trials. For example, for in vivo injection, such as injection directly into the eye of a subject, an effective dose can be from about 105 to about 1015 lentiviral or AAV virions, for example, from about 106 to about 1012 virions, about 108 to 1012 virions, or about 108 to 1015 virions. Other effective dosages can be readily established by one of ordinary skill in the art through routine trials establishing dose response curves.

Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

Other factors can include, e.g., other medical disorders concurrently or previously affecting the subject, the general health of the subject, the genetic disposition of the subject, diet, time of administration, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

### II. Compositions

Also provided herein is a vector comprising a nucleic acid sequence encoding a miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5). In some embodiments, the vector comprises a miRNA that at least 95% identity to miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), or a miR-146b precursor (SEQ ID NO: 5). In some embodiments, the vector comprises multiple copies, i.e., two or more, three or more, four or more, five or more, etc., of each of the one or more miRNAs.

In some embodiments, the vector is a viral vector. In some embodiments, the vector is a DNA vector or RNA vector. Examples of viral vectors include, but are not limited to, an adeno-associated virus (AAV) vector, a retroviral vector, a lentiviral vector, a herpes simplex viral vector, or an adenoviral vector. It is understood that any of the viral vectors described herein can be packaged into viral particles or virions for administration to the subject.

In some embodiments, the viral vector is an AAV vector comprising a 5′ inverted terminal repeat and a 3′ inverted terminal repeat. In some embodiments, the AAV vector can be a single-stranded AAV vector or a self-complementary AAV vector. Examples of AAV vectors include AAV1, AAV2, AAV3, AAV4, AAV5, AAV6, AAV7, AAV8, AAV9, AAV10, AAV11 and AAV12. In some embodiments, the viral vector is an AAV vector with a capsid protein of an AAV selected from the group consisting of AAV1, AAV2, AAV3, AAV4, AAV5, AAV6, AAV7AAV8, AAV9, AAV10, AAV11 and AAV12. Methods for producing AAV vectors are known in the art. See for example, Clement and Greiger, “Manufacturing of recombinant adeno-associated viral vectors for clinical trial,” Mol. Ther. Methods & Clinical Development 3: 16002 (2016); and Fripont et al. “Production, Purification, and Quality Control for Adeno-associated Virus-based vectors,” J. Vis. Exp. (143), e58960 (2019).

In some embodiments, the viral vector is a lentiviral vector. Methods for making lentiviral vectors are known in the art. See, for example, Wang and McManus “Lentivirus production,” J. Vis. Exp. 32: 1499 (2009); and Tiscomia et al. “Production and purification of lentiviral vectors,” Nature Protocols 1: 241-245 (2006).

In some embodiments, the one or more miRNAs or the DNA sequence encoding the one or more miRNAs are operatively linked to a promoter. In some embodiments, the promoter is a constitutive promoter, for example, a cytomegalovirus (CMV) promoter, a human elongation factor-1 alpha (EF1 α) promoter, a CMB enhancer fused to the chicken beta-actin promoter (CAG promoter), a phospholycerate kinase (PGK) promoter, or a U6 promoter. In some embodiments the CMV promoter comprises a sequence that has at least 95% identity to SEQ ID NO: 8. In some embodiments the CAG promoter comprises a sequence that has at least 95% identity to SEQ ID NO: 9.

Inducible promoters such as the tetracycline inducible promoter or a glucocorticoid inducible promoter can also be used. The nucleic acids described herein can also be under the control of a tissue-specific promoter to promote expression of the nucleic acid in specific cells, tissues or organs. In some embodiments, the promoter comprises an eye tissue-specific promoter.

Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

In some embodiments, the vector comprises multiple, i.e., two or more, three or more, four or more, five or more etc., of each miRNA or DNA sequence encoding an miRNA.

In some embodiments, a vector also comprises an antisense molecule, under the control of an inducible promoter, wherein the antisense molecule targets the promoter driving expression of the one or more miRNAs.

Also provided is a cell comprising any of the vectors described herein. The host cell can be an in vitro, ex vivo, or in vivo host cell. Populations of any of the host cells described herein are also provided. A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook, Ausubel, and Berger (all supra), as well as Freshney (1994) Culture of Animal Cells, a Manual of Basic Technique, 3rd Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4th Ed. W.H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be an HEK293T cell, a Chinese hamster ovary (CHO) cell, a COS-7 cell, a HELA cell, an avian cell, a myeloma cell, a Pichia cell, an insect cell or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host.

Methods for introducing vectors into cells are known in the art. As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (for example, a CRISPR/Cas9 system), a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid into a host cell

Also provided is a nanoparticle comprising a miRNA or a DNA sequence encoding an miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5). In some embodiments, the nanoparticle comprises an miRNA that has at least 95% identity to an miRNA selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5). See, for example, Lee et al. “MicroRNA delivery through nanoparticles,” Journal of Controlled Release, “313(10): 80-95 (2019); Zhou et al. “Nanoparticles in the ocular drug delivery,” Int. J. Ophthalmol. 6(3): 390-396 (2013); and Omerovic and Vranic, “Application of nanoparticles in ocular drug delivery systems,” Health and Technology 10:61-78 (2020).

Also provided is a pharmaceutical composition comprising any of the nucleic acids, vectors or nanoparticles described herein. The pharmaceutical compositions can also comprise a pharmaceutically acceptable carrier, diluent, excipient, or buffer. In some embodiments, the pharmaceutically acceptable carrier, diluent, excipient, or buffer is suitable for use in a subject, for example, a human. The pharmaceutical compositions can be delivered to a subject, so as to allow production of an expression product in the eye of the subject and produce an effective amount of an expression product that reduces IOP or treats glaucoma.

The compositions may be administered alone or in combination with at least one other agent, such as stabilizing compound, which may be administered in any sterile, biocompatible pharmaceutical carrier, including, but not limited to, saline, buffered saline, dextrose, and water. In some embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Loyd V. Allen et al, editors, Pharmaceutical Press (2012).

### Exemplary Embodiments

Exemplary embodiments of the invention include:

1.A method for decreasing intraocular pressure (IOP) in a subject comprising: increasing expression of one or more microRNAs (miRNAs) in the eye of a subject in need thereof, wherein the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

2. A method for treating glaucoma in a subject comprising increasing expression of one or more microRNAs (miRNAs) in the eye of a subject in need thereof, wherein the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

5. The method of embodiment 1 or 2, wherein expression of the one or more miRNAs is increased by administering to the eye of a subject in need thereof one or more miRNAs having at least 95% identity to a nucleic acid sequence selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

5. The method of any one of embodiments 1-3, wherein one or more DNA sequences that encode the one or more miRNAs are administered to the eye of the subject.

5. The method of any one of embodiments 1-4, wherein expression of the one or more miRNAs is increased in one or more cells of the outflow pathway of the subject.

6. The method of embodiment 5, wherein the one or more cells are selected from the group consisting of a trabecular meshwork cell, a Schlemm's canal cell, and a juxtacanalicular cell.

7. The method of any one of embodiments 3-6, wherein the one or more miRNAs or the one or more DNA sequences encoding the one or more miRNAs is in a viral vector.

8. The method of embodiment 7, wherein the viral vector is an adeno-associated viral vector or a lentiviral vector.

9. The method of embodiment 7 or 8, wherein the one or more miRNAs are operatively linked to a promoter.

10. The method of embodiment 9, wherein the promoter comprises a CAG promoter sequence.

11. The method of embodiment 9, wherein the promoter comprises an eye tissue-specific promoter.

12. The method of any one of embodiments 3-6, wherein the one or more miRNAs or the one or more DNA sequences encoding the one or more miRNAs is administered to the eye of the subject via non-viral delivery.

13. The method of embodiment 12, wherein non-viral delivery comprises nanoparticle delivery.

14. The method of any one of embodiments 1-13, wherein the method further comprises administration of one or more agents selected from the group consisting of a prostaglandin analog, a beta blocker, an alpha-adrenergic agonist, a carbonic anhydrase inhibitor, and a cholinergic agent.

15. The method of any one of embodiments 1-14, wherein the subject has had eye surgery to reduce IOP.

16. The method of any one of embodiments 1-14, wherein the method further comprises conducting eye surgery on the subject to reduce IOP.

17. A vector comprising a nucleic acid sequence encoding a miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

18. The vector of embodiment 17, wherein the vector is a viral vector.

19. The vector of embodiments 17 or 18, wherein the vector is a DNA vector or RNA vector.

20. The vector of embodiment 19, wherein the vector is an adeno-associated viral vector or lentiviral vector.

21. A cell comprising the vector of any one of embodiments 18-20.

22. A nanoparticle comprising a miRNA or a DNA sequence encoding an miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

23. A pharmaceutical composition comprising the vector of any one of embodiments 17-20 or the nanoparticle of embodiment 22.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

It is difficult to identify a target for sustained decrease in IOP. Unsuccessful attempts to identify a target for sustained decrease in IOP in this laboratory include overexpression and inhibition of microRNAs of the miR-200, miR-29, and cluster miR-143/145 familes. In addition, overexpression of interleukin 6 (IL6) matrix gla protein (MGP), chitinase 3 like 1 (CHI3L1), p16INK4a was attempted. CRISPR/Cas9 mediated inhibition of formin homology 2 domain containing 1 (FHOD1), lysophosphatidic acid receptor 1 (LPAR1/EDG2), and endothelin A receptor (ETAR) was also performed.

Previous studies showed that miR-146 was upregulated in senescent fibroblasts and trabecular meshwork cells and, that its upregulation could act as a brake to excessive production of inflammatory cytokines that are part of the senescence associated secretory phenotype (Li et al. “Modulation of inflammatory markers by miR-146a during replicative senescence in trabecular meshwork cells,” Invest. Ophthalmol. Vis. Sci. 51(6): 2976-2985 (2010)). Additional studies showed that miR-146 was up-regulated by cyclic mechanical stress in trabecular meshwork (TM) cells (Luna et al. “MicroRNA-24 regulates the processing of latent TGFβ1 during cyclic mechanical stress in human trabecular meshwork cells through direct targeting of FURIN,” J. Cell Physiol. 226(5): 1407-1414 (2011)).

Previous unsuccessful attempts to reduce IOP involved modulation of Interleukin 6 (IL6), Chitinase 3 like 1 (CH3L1), Transforming growth factor beta 1 (TGFβ1), and/or lysophosphatidic acid receptor 1 (LPAR1/EDG2) expression. Given that production of some inflammatory cytokines by TM cells in response to mechanical stress had been shown to increase aqueous humor outflow facility, it was reasoned that induction of miR-146a could act as a brake to prevent excessive production of cytokines in conditions of elevated intraocular pressure.

Cell Culture, miRNA Transfection, and Virus Transduction

Human trabecular meshwork (HTM) primary cell cultures were generated from cadaver eyes, or cornea rings with no history of eye disease. Cell cultures were maintained at 37° C. in 5% CO2 in media (low glucose Dulbecco's Modified Eagle Medium with L-glutamine, 110 mg/ml sodium pyruvate, 10% fetal bovine serum, 100 mM non-essential amino acids, 100 units/ml penicillin. All reagents were obtained from Thermo-Fisher Scientific (Waltham, Mass.). HTM primary cells, passage 3-4 (HTM L3, age 25 and HTM 1788, age 23) were transfected, at around 70% confluency, one day after plating, using lipofectamine 2000 (Thermo-Fisher Scientific), following manufacturer's instructions. Cells were transfected with hsa-miR-146a mimic (146M), hsa-miR-146a inhibitor (146I), negative control mimic or inhibitor (scramble), at 40 pmol concentration (Dharmacon, Chicago, Ill.) and analyzed 72 hours after transfection. HTM cells transduced with adenovirus (CAG miR-146a and CAG-empty, 50 pfu each) or lentivirus (CAG-146a, 30 pfu), were analyzed 72 hours and 1 week after transduction, respectively.

### Cyclic Mechanical Stress (CMS)

HTM cell cultures were plated on type I collagen-coated flexible silicone bottom plates (Flexcell, Hillsborough, N.C.), and transfected 24 hours later with miRNAs. Cells were subjected to CMS 72 hours after transfection. Medium was switched to serum-free DMEM two hours before CMS and cells were stretched for 4 hours (20% stretching, 1 cycle per second), using the computer-controlled, vacuum-operated FX-3000 Flexercell Strain Unit (Flexcell, Hillsborough, N.C.). A frequency of 1 cycle per second was selected to mimic cardiac frequency. Control cells were cultured under the same conditions, but no mechanical force was applied.

Quantitative PCR (qPCR)

Total RNA was isolated from HTM cells and from the anterior chamber (i.e cornea, trabecular meshwork, ciliary body and a small fraction of iris and sclera) of rat eyes using Direct-zol RNA MiniPrep Kit (ZymoResearch, Irvine, Calif.); according to manufacturer's instructions. RNA yields were measured using nanodrop (Thermo Fisher Scientific). First strand cDNA was synthesized from total RNA (700 ng cells and 500 ng tissue) by reverse transcription using oligodT and Superscript II reverse transcriptase (Invitrogen, Carlsbad, Calif.) according to manufacturer's instructions. q-PCR reactions were performed in a 20 μl mixture containing 1 μl of the cDNA preparation, 1× iQ SYBR Green Supermix (Bio-Rad, Hercules, Calif.), using the following PCR parameters: 95° C. for 3 minutes followed by 40 cycles of 95° C. for 15 seconds, 55° C. for 15 seconds and 72° C. for 15 seconds. Beta actin and GADPH were used as internal normalized reference for cDNA. miRNA cDNAs (50 ng) were transcribed using a TaqMan microRNA reverse transcription kit and Taqman microRNA assays for miR-146a and RNU6B (normalized reference); and amplified using TaqMan® Universal PCR Master Mix (Applied Biosystems, Foster City, Calif.), following manufacturer's instructions. The absence of nonspecific products was confirmed by melt curves analysis. All qPCR experiments were performed with a CFX96 Thermal Cycler (Bio-Rad). Results from qPCR experiments are expressed as 2—ΔCt (delta CT) and represented as a percentage of the control ±s.d.; or 2—ΔΔCt (fold) ±s.d. The primers used for q-PCR amplification are shown in Table 1 and were designed using Primer 3 (Untergasser A et al., “SG Primer—new capabilities and interfaces,” Nucleic Acids Research 40(15):e115 (2012).

TABLE 1
q-PCR amplification primers

| Target | NCBI | | |
|---|---|---|---|
| name | gene ID | Forward 5′ - 3′ | Reverse 5′ - 3′ |
| Hs. COX1 | ID: 5742 | TAGAGATTGGGGCTCCCTTT | AGGGACAGGTCTTGGTGTTG |
| | | (SEQ ID NO: 15) | (SEQ ID NO: 30) |
| Hs COX2 | ID: 5743 | TGAGCATCTACGGTTTGCTG | TGCTTGTCTGGAACAACTGC |
| | | (SEQ ID NO: 16) | (SEQ ID NO: 31) |
| Hs. IL1b | ID: 3553 | AACAGGCTGCTCTGGGATTCTCTT | ATTTCACTGGCGAGCTCAGGTACT |
| | | (SEQ ID NO: 17) | (SEQ ID NO: 32) |
| Hs. IL8 | ID: 3576 | AGAAACCACCGGAAGGAACCATCT | CACCTTCACACAGAGCTGCAGAAA |
| | | (SEQ ID NO: 18) | (SEQ ID NO: 33) |
| Hs. IL6 | ID: 3569 | AAA TTCGGTACATCCTCGACGG | AGTGCCTCTTTGCTGCTTTCACAC |
| | | (SEQ ID NO: 19) | (SEQ ID NO: 34) |
| Hs. Serpine | ID: 5054 | AATGTGTCATTTCCGGCTGCTGTG | ACATCCATCTTTGTGCCCTACCCT |
| 1 | | (SEQ ID NO: 20) | (SEQ ID NO: 35) |
| Hs.IRAK1 | ID: 3654 | ATTTATGCTTGGGAGGTCGAGGCT | TCGCTTCTTGCTAGGACTGAACCA |
| | | (SEQ ID NO: 21) | (SEQ ID NO: 36) |
| Hs. HSP70 | ID: 3310 | ACAGGAGCACAGGTAAGGCT | TTCATGAACCATCCTCTCCA |
| | | (SEQ ID NO: 22) | (SEQ ID NO: 37) |
| Hs. | ID: 2597 | TCAACAGCGACACCCACTCCTC | ATGAGGTCCACCACCCTGTTGC |
| GADPH | | (SEQ ID NO: 23) | (SEQ ID NO: 38) |
| Hs. beta | ID: 60 | CCTCGCCTTTGCCGATCCG | GCCGGAGCCGTTGTCGACG |
| actin | | (SEQ ID NO: 24) | (SEQ ID NO: 39) |
| Rn. | ID: | GGTCCCAACAAGGAGGAGAA | GCTTGGTGGTTTGCTACGAC |
| TNFalpha | 24835 | (SEQ ID NO: 25) | (SEQ ID NO: 40) |
| Rn. IL1b | ID: | CACTCATTGTGGCTGTGGAG | AGGACGGGCTCTTCTTCAAA |
| | 24494 | (SEQ ID NO: 26) | (SEQ ID NO: 41) |
| Rn. CD68 | ID: | ACGGACAGCTTACCTTTGGA | AATGTCCACTGTGCTGCTTG |
| | 287435 | (SEQ ID NO: 27) | (SEQ ID NO: 42) |
| Rn. | ID: | AAGATGGTGAAGGTCGGTGT | GCTTCCCATTCTCAGCCTTG |
| GADPH | 24383 | (SEQ ID NO: 28) | (SEQ ID NO: 43) |
| Rn. beta | ID: | TCCTCCCTGGAGAAGAGCTA | ACGGATGTCAACGTCACACT |
| actin | 81822 | (SEQ ID NO: 29) | (SEQ ID NO: 44) |

Recombinant Adenovirus, Lentivirus and Adeno-Associated Virus (AAV), Preparation

To prepare adenovirus miR-146a under control of the CAG-promoter, pENTR1A was modified to eliminate the restriction sites for Not1 and EcoR1 from the multiple cloning site by PCR amplification using the following primers F: 5′-AAAAGCTTGCCGCACTCGAGATATCTAGACC3′ (SEq ID NO: 10) and R:5′ CGGTACCGGATCCAGTCGACTGAATTGGTTC3′ (SEQ ID NO: 11) (restriction sites are in bold) followed by digestion of the PCR product with SalI and HindIII and ligation to the fragment generated from the digestion of the pCAGEN plasmid with SalI-HindIII. The fragment from pCAGEN contains the CAG promoter, multiple cloning site (EcoRi/HhoI/EcorV/NotI), and a rabbit globin polyA signal (Addgene, Cambridge Mass. plasmid #11160, Matsuda et al, “Electroporation and RNA interference in the rodent retina in vivo and in vitro,” Proc Natl Acad Sci USA. 2004; 101(1):16-22. The miR-146a was obtained through PCR amplification of the pre-mirna or precursor region from plasmid 15092 (Addgene, Taganov K D et al., “NF-kappaB-dependent induction of microRNA miR-146, an inhibitor targeted to signaling proteins of innate immune responses,’ Proc Natl Acad Sci U S A. 2006; 103(33):12481-6) using the following primers: F 5′-TGGTCTCTAATT GGCTGGGACAGGCCTGGAC-3′ (SEQ ID NO: 12) and R 5-AGGCGGCCGCTCGAGGAGCCTGAGACTCTG-3′ (SEQ ID NO:13) (restriction sites are in bold, BsaI with EcoRI end and Not1). miR-146a PCR product was cloned into pENTR1A-CAG digested with EcoRI-NotI to generate pENTR-CAG-146a; miR-146a was confirmed by sequencing. Recombinant adenovirus for miR-146a and empty plasmid (pENTR1A-CAG) were prepared using the Getaway System from Invitrogen, following manufacturer's instructions; CAG-mir-146a/pENTR1A or CAG-/pENTR1A were recombined with pAd/PL-DEST (Invitrogen) using LR recombinase (Invitrogen). miR 146 plasmid was re-sequencing for confirmation. Adenoviruses were amplified, purified and titer determined using the Adenovirus mini purification Kit (VIRAPUR, San Diego, Calif.) and Adeno-X Rapid Titer Kit (Clontech, Mountain View, Calif.) respectively, following manufacturer's instructions.

For AAV preparation, the PCR fragment of miR-146 was digested with BsaI (with compatible EcoRI end) and Not1, and cloned between the BamHI and Not1 sites of pscAAV-CAG-GFP plasmid (Addgene #83279, Pekrun et al., “Using a barcoded AAV capsid library to select for clinically relevant gene therapy vectors,” JCI Insight. 2019; 4 (22); this resulted in replacement of the EGFP gene with miR-146a to generate the pscAAV-CAG-miR-146a plasmid. To generate lentivirus expressing miR-146a under the control of the CAG promoter, the IRIS-GFP fragment from vector plenti-CAG-IRES-GFP (Addgene Plasmid #69047 Labidi-Galy S I et al., “Elafin drives poor outcome in high-grade serous ovarian cancers and basal-like breast tumors,” Oncogene. 2015 Jan. 15; 34(3):373-83) was replaced with miR-146a, by digestion of plenti-CAG-IRES-GFP with EcoRI and Not1 and miR-146 with BsaI with EcoRI compatible end and Not1.

Both lentivirus and AAV were prepared at Duke University School of Medicine Viral Vector Core. Lentivirus vector is based on the human immunodeficiency virus (HIV) pseudotyped with the vesicular stomatitis virus G-protein (VSV-G). AAV viruses (miR-146 and GFP) are scAAV type 2 vector with miR-146a and GFP driven by the same CAG promoter.

### In Vivo Injections, IOP Measurements and Ophthalmic Examinations

All experiments with animals were conducted in accordance with the ARVO statement for the use of Animals in Ophthalmic and Vision Research and previously approved by the Institutional Animal Care and Use Committee at Duke University. Brown-Norway females retiree breeder rats (Charles River Laboratories, Wilmington, Mass.), were housed in pairs with a 12 hour cycle of light and dark; and water and food ad libitum. For intraocular injections, rats were anesthetized with a mixture of oxygen and isofluorane (V-1 Table Top System, VetEquip, California) plus a drop of topical anesthetic (proparacaine hydrochloride, Akorn Pharmaceuticals, New Jersey). Rats were injected with viral suspension in one eye using insulin syringes with ultra-fine needle (31G) (BD Biosciences, San Jose, Calif.), and the contralateral eye was used as a control. The needle was inserted through the peripheral cornea with bevel up and the bevel rotated 90 degrees for viral inoculation. Ten rats were injected with adenovirus CAG-146a and 6 rats with adenovirus CAG-GFP (both with 1.5×1011 pfu/μl, 15 μl), 10 rats with AAV-CAG-146a and 6 rats with AAV-GFP (both with 3×1013 pfu/ml, 15 μl), and 9 rats with lenti CAG-146a (3 rats with 5.6×108, 25 μl and 6 rats with 1.0×109 pfu/ml, 15 μl).

IOP was measured in awake animals using topical anesthetic, two times a week for the first 3 months and once a week after that. Measurements were taken at the same time of the day, between 8:30-10 a.m., with an average of six readings per eye, using a portable tonometer, (Tonolab, Helsinky, Finland).

Examination of the anterior chamber structures (corneal epithelium, endothelium, chamber depth, lens status, and inflammation) were performed by a board certified ophthalmologist using a slit lamp bio-microscope (Topcon) and an ophtalmoscope (Universal S3, Zeiss, Germany).

### Optomotor Response

Visual function was evaluated by opto-kinetic tracking response using OptoMotry (Cerebral Mechanics, Medicine Hat, Alberta, Canada). Briefly, rats were placed on an elevated platform in the middle of a chamber surrounded by screen monitors displaying vertical gratings rotating at a speed of 12 degrees/second. A camera situated above, images the behavior of the animal; and a cursor placed on the forehead of the animal allow the observer to track the head movement, clockwise (left eye) or counter clockwise (right eye) in response to the rotating gratings, in real time. To determine the spatial frequency threshold, the vertical bands were displayed at 100% contrast starting at 0.042 cycles/degree and the spatial frequency was increased until the animal no longer tracks.

### Semi-Thin Sections

Control (not injected) and experimental (lenti CAG-miR-146a injected) eyes were enucleated, washed in PBS and fixed in 2% glutaraldehyde/2% paraformaldehyde in PBS; post-fixed in 1% osmium tetroxide, dehydrated, embedded in epoxy, sectioned (0.5 μm) and stained at the Morphology Facility at Duke University Eye Center. Semi-thin sections were examined by light microscopy (Axioplan 2, Zeiss, Germany).

### Statistics

Paired Student's t-test was used for IOP comparison between lenti-CAG-miR-146a, Ad.-CAG-miR-146a, and Ad.-CAG-empty and their respective contralateral control (non-injected) eyes. Data are presented as mean±s.d. Results from q-PCR are represented as a percentage of the control ±s.d.; or fold ±s.d.; significance for q-PCR was evaluated using unpaired Student's t-test. P value of <0.05 was considered significant.

### Results

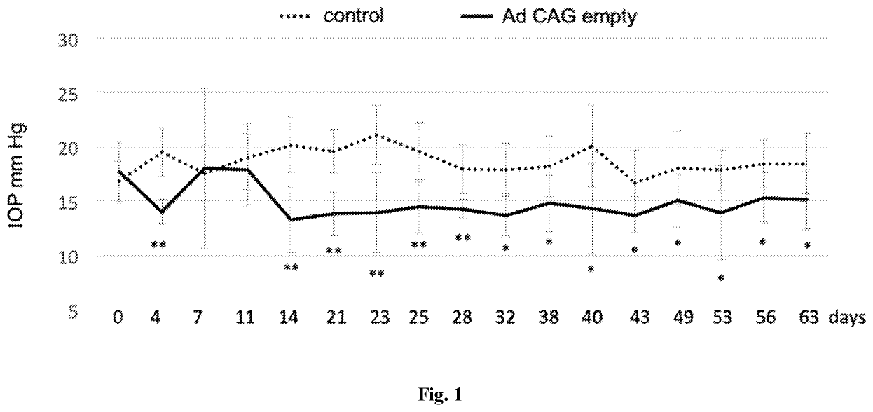

As set forth above, based on previous studies with miR146a, it was anticipated that overexpression of miR-146 in the TM could result in an increase of IOP. To test this hypothesis, an adenoviral vector expressing miR-146a under the control of a cytomegalovirus (CMV) enhancer fused to the chicken beta-actin promoter (CAG) promoter system (pCAGEN, Addgene Plasmid #11160, Watertown, Mass.) was constructed. The human miR-146a gene sequence (SEQ ID NO: 5) encoding a percursor RNA (set forth in Table 2), was obtained from Addgene Plasmid #15092 (Taganov et al. NF-kappB-dependent induction of microRNA miR-146, an inhibitor targeted to signaling proteins of innate human responses,” PNAS 103(33): 12581-6 (2006)). A mature human miR-146a-5′ (SEQ ID NO: 1) and a human mature 146a-3′ sequence (SEQ ID NO: 2), as set forth in Table 2, were expressed from this precursor miRNA. 7.5×107 pfus of the adenoviral vector were injected into the anterior chamber of one eye in each of ten brown Norway rats. Surprisingly, overexpression of miR-146a resulted in a sustained decrease in IOP (Average difference with contralateral eyes=4.024075397 mmHg ±1.993256187) that lasted at least two months, which is when rats were sacrificed (FIG. 1). Further, adverse effects to the cornea, such as neovascularization, opacification and edema were not observed when miR-146a was administered, thus showing the administration of miR-146a is well-tolerated by the cornea. Cataracts, conjunctival hyperemia, aqueous flare, and adverse changes in anterior chamber cells and vitreal cavity cells were also not observed.

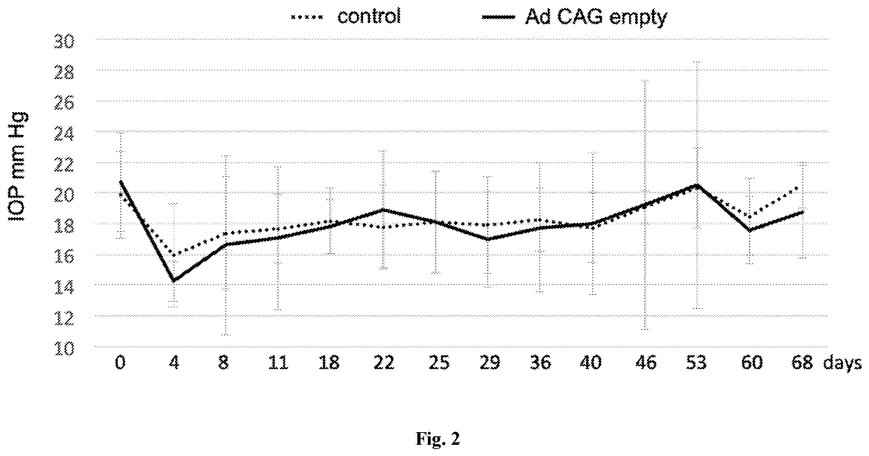

To ensure that the effects on IOP were not a non-specific effect of the adenoviral construct with the CAG promoter system, we injected one eye in each of six rats with a control virus under the control of the same CAG promoter system, but without an insert. As shown in FIG. 2, no significant change in IOP was observed between injected and non-injected contralateral control eyes (Average difference=0.35897619 mmHg ±0.84573066; n=6).

Since adenoviral vectors are not ideal for gene therapy, an scAAV type 2 vector (as described in Borras et al. “Mechanism of AAV transduction in glaucoma-associated human trabecular meshwork cells,” J. Gene Med. 8(5): 589-602 (2006)), including miR-146a, driven by the CAG promoter, as well as a control scAAV type 2 expressing GFP under the control of the same promoter, were tested.

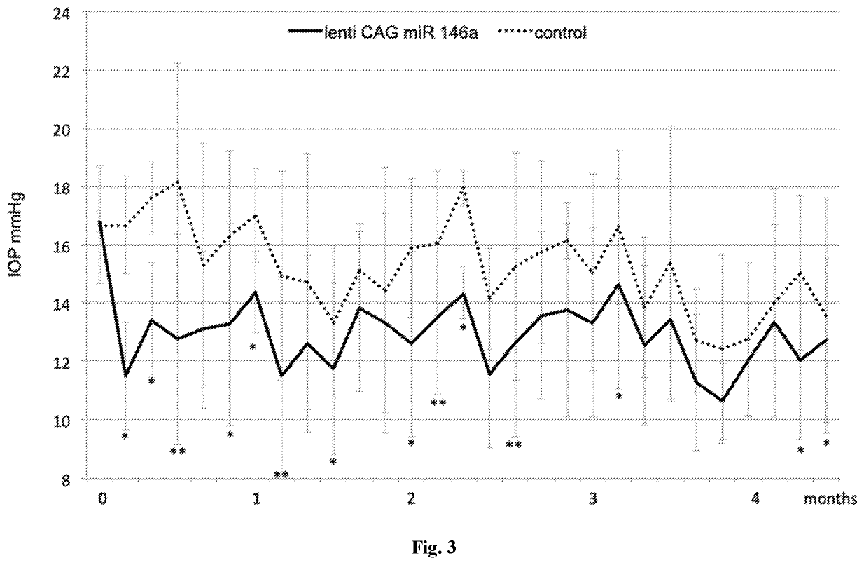

Injection of these vectors, at up to 1010 pfu, in the anterior chamber of living rats, did not result in any measurable effect in IOP, and expression of GFP was limited to a very small number of cells in the TM. Therefore, lentiviral vectors expressing miR-146a, under the control of the CAG promoter, were generated by replacing the IRIS-GFP fragment from vector plenti-CAG-IRES-GFP (Addgene Plasmid #69047) with miR-146a. A first preparation of this lentiviral vector yielded 5.6×108 pfu/mL. Given the relatively low titer, 25 μL were injected in the anterior chamber of three brown Norway rats. Injections with such high volume are likely to result in loss of viral particles that will exit the anterior chamber due to high pressure and stretching of the TM. It is therefore difficult to evaluate the level of transduction of TM cells in these conditions. However, as shown in FIG. 3, injected eyes showed a decrease in IOP that lasted more than 3 months.

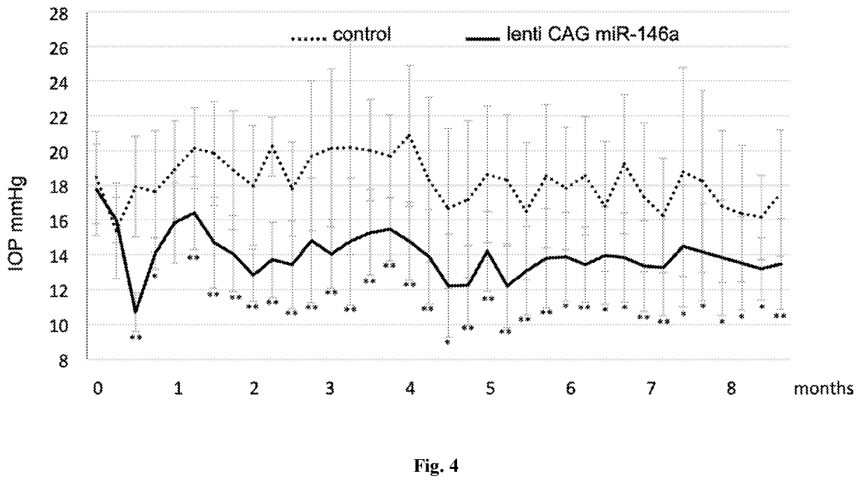

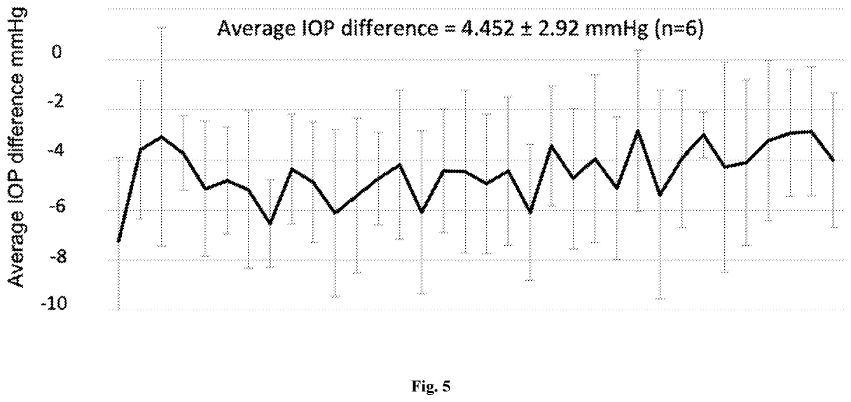

A second preparation of the same lentiviral vector yielded a titer of 1×109 pfu/mL. With this preparation, six eyes were injected with a total volume of 15 μL per eye (1×109 pfu/mL). The effects on IOP of these injections were of higher magnitude compared to the previous set of injections and were sustained in all animals for six months. For 5 our of 6 animals, the effect was sustained for more than 8 months (FIG. 4), with an average difference in IOP of 4.452±2.928, n=6 (FIG. 5).

Eyes Injected with miR-146a Exhibit Normal Function and Morphology

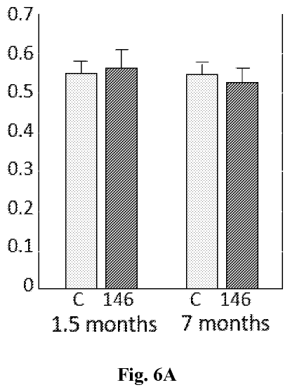

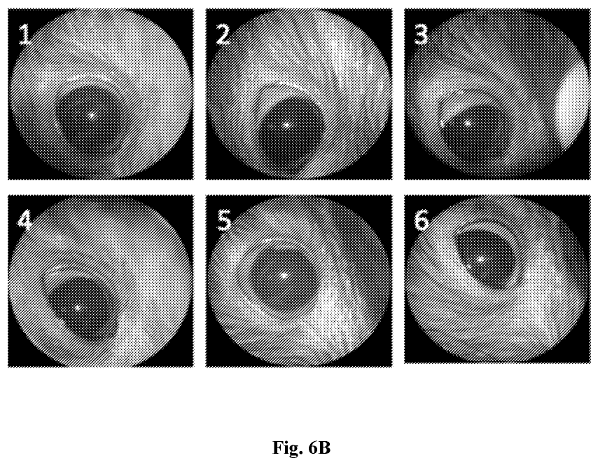

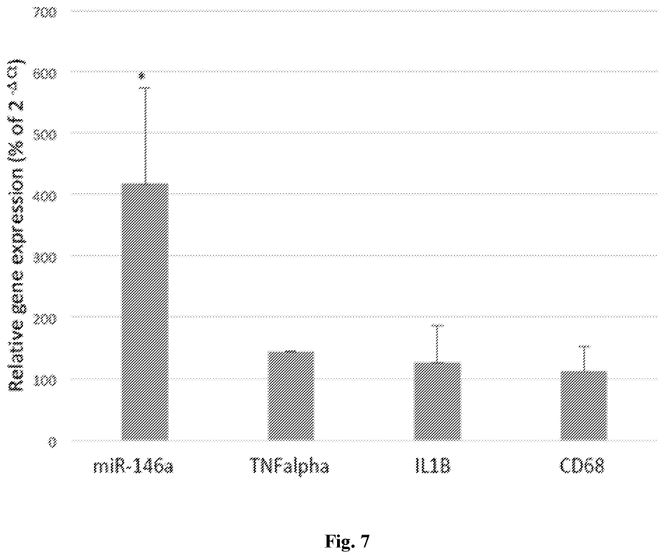

To evaluate whether treatment with lentivirus expressing CAG-miR-146a had any potential adverse effect in eye function visual acuity by analyzed by involuntary image-tracking (optokinetic reflex) in an OptoMotry system, at one-and-a half, and at seven months after injection. The results showed no significant difference in visual acuity between treated (146) and control (C) eyes (FIG. 6A). Visual inspections of the eyes did not reveal any obvious abnormalities, such as opacity or corneal edema, at any time during the experiment. Slit-lamp and ophthalmoscope examinations for a certified ophthalmologist at 6 and 7 months reveals no visible abnormalities or signs of inflammation associated with lentivirus delivery of miR-146a (FIG. 6B). Inflammation and expression of miR-146a were also evaluated post-mortem by q-PCR. TNF alpha, CD68, IL1B and miR-146a were analyzed in the anterior chamber of three animals. miR-146a was significantly upregulated (Fold: 4.281±1.970; p-value: 0.0015) in the eyes injected with lenti CAG-miR-146, nine months after injection; inflammatory genes were not significantly different compared to no injected eyes (FIG. 7).

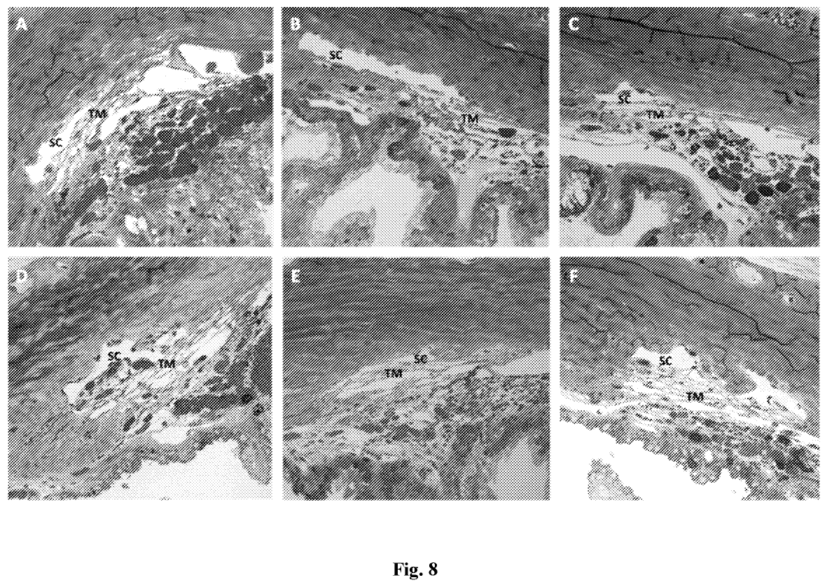

Semi-thin sections analysis of the angle of three rat eyes showed no apparent abnormalities on the eyes injected with lenti-miR-146 and not evident differences with the contralateral control eyes (FIG. 8)

In addition, two HTM primary cell lines were transduced with an miR-146a mimic (ugagaacugaauuccauggguu) (SEQ ID NO: 1), an miR-146 inhibitor, which is the antisense of mature miR-146a (aacccauggaauucaguucuca) (SEQ ID NO: 14), and negative control scrambles; and subjected to cyclic mechanical stress (CMS) or kept under the same conditions, but no mechanical force was applied. Nine genes (COX2, IL6, HSP70, IRAK1, BMP2, PAIL COX1, IL8), chosen because they were up- or down-regulated by CMS and/or miR146 in previous experiments, were analyzed.

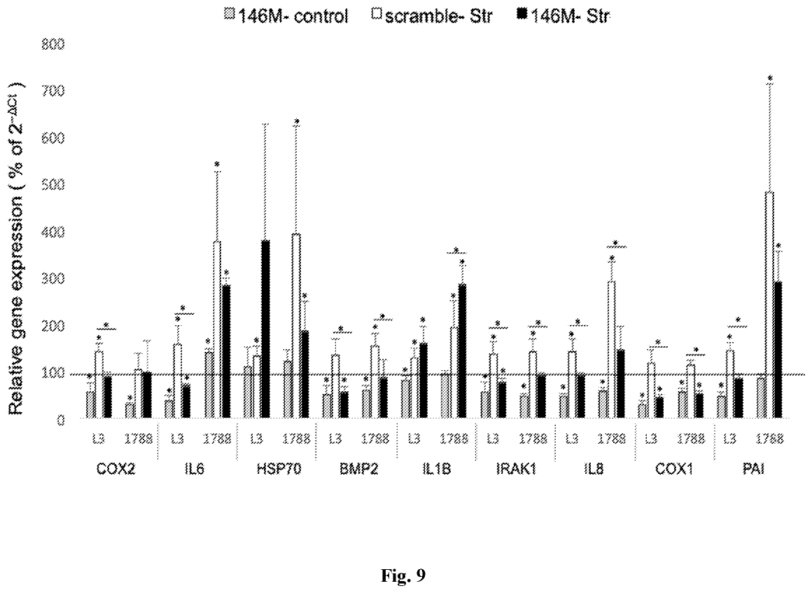

HTM cells transduced with an miR146 mimic, showed significant down regulation for most of the analyzed genes, with an average of 34% reduction in gene expression, compared to negative control scramble. When cells transduced with the miR146a mimic or negative control scramble were subjected to CMS, the induction of gene expression due to the stretching was partially or totally inhibited in miR146a-transduced cells, for most of the analyzed genes. After CMS control scramble increased gene expression an average of 93% and 146a mimic by 42%, in comparison to scramble non-stressed (FIG. 9).

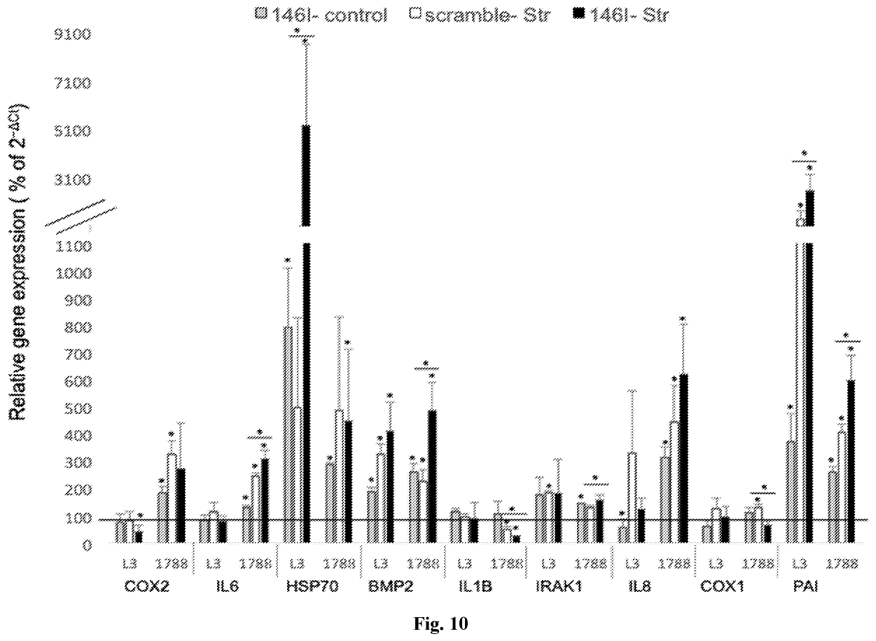

HTM cells transduced with miR-146 inhibitor increase the basal expression levels of the analyzed genes in average 106% compared to negative control. When subjected to CMS, cells transfected with an miR-146 inhibitor increases the expression an average of 446% when compared to scramble control subjected to CMS, but this increase was not as homogeneously distributed among genes as was with an miRNA-146a mimic (FIG. 10).

As set forth above, the initial hypothesis was that miR-146 could act as a brake in the production of inflammatory cytokines. Given that production of some inflammatory cytokines by TM cells in response to mechanical stress had been shown to increase aqueous humor outflow facility (Liton et al., “Stress response of the trabecular meshwork.” Journal of glaucoma 2008; 7 (5): 378-85), it was hypothesized that the induction of miR-146a could elevate IOP. Previous experiments using a different construct of miR-146, (influenza associated virus-CMV promoter-mature mirna) produced a transient increase in IOP in rats (Luna et al., “Role of miR-146a in the Regulation of Gene Expression Changes Induced by Mechanical Stress in Trabecular Meshwork Cells,” Invest. Ophthalmol. Vis. Sci. 2014; 55(13):4516). Therefore, the decrease in IOP observed upon increasing expression of miR-146A in the eye, was surprising and unexpected.

Further, eyes transduced with miR-146a showed no signs of inflammation, likely because of its anti-inflammatory effect, but also because miRNAs showed low toxicity and immunogenicity when compared to DNA gene therapy or protein-based drug molecules (Chen et al., 2015). In vivo examination and analysis of anterior chamber sections exhibited no obvious signs of inflammation in miR-146 transduced or control eyes (non-injected). Although a lentivirus control was not used for comparison with miR-146a, lentiviruses (HIV or FIV based) have been successfully inoculated into the anterior chamber of rats, cats and monkeys without significant inflammatory reactions or effects on IOP; and lentivirus expressing GFP has been shown to transduce mainly to the trabecular meshwork (See for example, Tan et al. “Effects of Lentivirus-Mediated C3 Expression on Trabecular Meshwork Cells and Intraocular Pressure,” Invest Ophthalmol Vis Sci. 2018 Oct. 1; 59(12):4937-4944; and Xiang et al., “Gene transfer to human trabecular meshwork cells in vitro and ex vivo using HIV-based lentivirus.” Int J Ophthalmol. 2014 Dec. 18; 7(6):924-9.)

Overall, these results show that administration to the cells of the anterior chamber of a mammalian subject, of a viral vector expressing miR-146a, can decrease IOP without obvious adverse effects, for more than eight months.

It is understood that the examples and embodiments described herein are for illustrative purposes only and that various modifications or changes in light thereof will be suggested to persons skilled in the art and are to be included within the spirit and purview of this application and scope of the appended claims.

TABLE 2
miRNA Sequences

| | MIRBase | | |
|---|---|---|---|
| miRNA | Accession No. | Sequence | SEQ ID NO: |
| Mature human miR-146a-5p | MIMAT0000449 | ugagaacugaauuccauggguu | 1 |
| Mature human miR-146a-3p | MIMAT0004608 | ccucugaaauucaguucuucag | 2 |
| Mature human miR-146b-5p | MIMAT0002809 | ugagaacugaauuccauaggcug | 3 |
| Mature human miR-146b-3p | MIMAT0004766 | gcccuguggacucaguucuggu | 4 |
| Human miR-146a precursor | | AATTGGCTGGGACAGGCCTGGACTGCA | 5 |
| encoding mature mir-146-5p | | AGGAGGGGTCTTTGCACCATCTCTGAAA | |
| (underlined) and mature mir- | | AGCCGATGTGTATCCTCAGCTTTGAGAA | |
| 146-3p (italicized) | | CTGAATTCCATGGGTTGTGTCAGTGTCA | |
| | | GACCTGTGAAATTCAGTTCTTCAGCTGGG | |
| | | ATATCTCTGTCATCGTGGGCTTGAGGAC | |
| | | CTGGAGAGAGTAGATCCTGAAGAACTT | |
| | | TTTCAGTCTGCTGAAGAGCTTGGAAGAC | |
| | | TGGAGACAGAAGGCAGAGTCTCAGGCT | |
| | | CCTCGAGCGGCC | |
| Human miR-146a stem loop | M10000477 | CCGAUGUGUAUCCUCAGCUUUGAGAAC | 6 |
| structure | | UGAAUUCCAUGGGUUGUGUCAGUGUC | |
| | | AGACCUCUGAAAUUCAGUUCUUCAGCU | |
| | | GGGAUAUCUCUGUCAUCGU | |
| Human miR-146b stem loop | M100031299 | CCUGGCACUGAGAACUGAAUUCCAUAG | 7 |
| structure | | GCUGUGAGCUCUAGCAAUGCCCUGUGG | |
| | | ACUCAGUUCUGGUGCCCGG | |

1. A method for decreasing intraocular pressure (IOP) in a subject comprising: increasing expression of one or more microRNAs (miRNAs) in the eye of a subject in need thereof, wherein the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

2. A method for treating glaucoma in a subject comprising increasing expression of one or more microRNAs (miRNAs) in the eye of a subject in need thereof, wherein the one or more miRNAs are selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

3. The method of claim 1, wherein expression of the one or more miRNAs is increased by administering to the eye of a subject in need thereof one or more miRNAs having at least 95% identity to a nucleic acid sequence selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5.

4. The method of claim 1, wherein one or more DNA sequences that encode the one or more miRNAs are administered to the eye of the subject.

5. The method of claim 1, wherein expression of the one or more miRNAs is increased in one or more cells of the outflow pathway of the subject.

6. The method of claim 5, wherein the one or more cells are selected from the group consisting of a trabecular meshwork cell, a Schlemm's canal cell, and a juxtacanalicular cell.

7. The method of claim 3, wherein the one or more miRNAs or the one or more DNA sequences encoding the one or more miRNAs is in a viral vector.

8. The method of claim 7, wherein the viral vector is an adeno-associated viral vector or a lentiviral vector.

9. The method of claim 7, wherein the one or more miRNAs are operatively linked to a promoter.

10. The method of claim 9, wherein the promoter comprises a CAG promoter sequence.

11. The method of claim 9, wherein the promoter comprises an eye tissue-specific promoter.

12. The method of claim 3, wherein the one or more miRNAs or the one or more DNA sequences encoding the one or more miRNAs is administered to the eye of the subject via non-viral delivery.

13. The method of claim 12, wherein non-viral delivery comprises nanoparticle delivery.

14. The method of claim 1, wherein the method further comprises administration of one or more agents selected from the group consisting of a prostaglandin analog, a beta blocker, an alpha-adrenergic agonist, a carbonic anhydrase inhibitor, and a cholinergic agent.

15. The method of claim 1, wherein the subject has had eye surgery to reduce IOP.

16. The method of claim 1, wherein the method further comprises conducting eye surgery on the subject to reduce IOP.

17. A vector comprising a nucleic acid sequence encoding a miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

18.-21. (canceled)

22. A nanoparticle comprising a miRNA or a DNA sequence encoding an miRNA, wherein the miRNA is selected from the group consisting of miR-146a-5p (SEQ ID NO: 1), miR-146a-3p (SEQ ID NO: 2), miR-146b-5p (SEQ ID NO: 3), miR-146b-3p (SEQ ID NO: 4), a miR-146a precursor (SEQ ID NO: 4), and a miR-146b precursor (SEQ ID NO: 5).

23. A pharmaceutical composition comprising the vector of claim 17.

24. A pharmaceutical composition comprising the nanoparticle of claim 22.


SEQUENCE LISTING (44 sequences)

SEQ ID NO: 1 - RNA, 22 nt, Artificial Sequence
  Synthetic construct
ugagaacugaauuccauggguu

SEQ ID NO: 2 - RNA, 22 nt, Artificial Sequence
  Synthetic construct
ccucugaaauucaguucuucag

SEQ ID NO: 3 - RNA, 23 nt, Artificial Sequence
  Synthetic construct
ugagaacugaauuccauaggcug

SEQ ID NO: 4 - RNA, 22 nt, Artificial Sequence
  Synthetic construct
gcccuguggacucaguucuggu

SEQ ID NO: 5 - DNA, 262 bp, Artificial Sequence
  Synthetic construct
aattggctgggacaggcctggactgcaaggaggggtctttgcaccatctctgaaaagccgatgtgtatcctcagctttgagaactgaattccatgggttgtgtcagtgtcagacctgtgaaattcagttcttcagctgggatatctctgtcatcgtgggcttgaggacctggagagagtagatcctgaagaactttttcagtctgctgaagagcttggaagactggagacagaaggcagagtctcaggctcctcgagcggcc

SEQ ID NO: 6 - RNA, 99 nt, Artificial Sequence
  Synthetic construct
ccgauguguauccucagcuuugagaacugaauuccauggguugugucagugucagaccucugaaauucaguucuucagcugggauaucucugucaucgu

SEQ ID NO: 7 - RNA, 73 nt, Artificial Sequence
  Synthetic construct
ccuggcacugagaacugaauuccauaggcugugagcucuagcaaugcccuguggacucaguucuggugcccgg

SEQ ID NO: 8 - DNA, 506 bp, Artificial Sequence
  Synthetic construct
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagag

SEQ ID NO: 9 - DNA, 584 bp, Artificial Sequence
  Synthetic construct
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 10 - DNA, 31 bp, Artificial Sequence
  Synthetic construct
aaaagcttgccgcactcgagatatctagacc

SEQ ID NO: 11 - DNA, 31 bp, Artificial Sequence
  Synthetic construct
cggtaccggatccagtcgactgaattggttc

SEQ ID NO: 12 - DNA, 31 bp, Artificial Sequence
  Synthetic construct
tggtctctaattggctgggacaggcctggac

SEQ ID NO: 13 - DNA, 30 bp, Artificial Sequence
  Synthetic construct
aggcggccgctcgaggagcctgagactctg

SEQ ID NO: 14 - RNA, 22 nt, Artificial Sequence
  Synthetic construct
aacccauggaauucaguucuca

SEQ ID NO: 15 - DNA, 20 bp, Artificial Sequence
  Synthetic construct
tagagattggggctcccttt

SEQ ID NO: 16 - DNA, 20 bp, Artificial Sequence
  Synthetic construct
tgagcatctacggtttgctg

SEQ ID NO: 17 - DNA, 24 bp, Artificial Sequence
  Synthetic construct
aacaggctgctctgggattctctt

SEQ ID NO: 18 - DNA, 24 bp, Artificial Sequence
  Synthetic construct
agaaaccaccggaaggaaccatct

SEQ ID NO: 19 - DNA, 22 bp, Artificial Sequence
  Synthetic construct
aaattcggtacatcctcgacgg

SEQ ID NO: 20 - DNA, 24 bp, Artificial Sequence
  Synthetic construct
aatgtgtcatttccggctgctgtg

SEQ ID NO: 21 - DNA, 24 bp, Artificial Sequence
  Synthetic construct
atttatgcttgggaggtcgaggct

SEQ ID NO: 22 - DNA, 20 bp, Artificial Sequence
  Synthetic construct
acaggagcacaggtaaggct

SEQ ID NO: 23 - DNA, 22 bp, Artificial Sequence
  Synthetic construct
tcaacagcgacacccactcctc

SEQ ID NO: 24 - DNA, 19 bp, Artificial Sequence
  Synthetic construct
cctcgcctttgccgatccg

SEQ ID NO: 25 - DNA, 20 bp, Artificial Sequence
  Synthetic construct
ggtcccaacaaggaggagaa

SEQ ID NO: 26 - DNA, 20 bp, Artificial Sequence
  Synthetic construct
cactcattgtggctgtggag

SEQ ID NO: 27 - DNA, 20 bp, Artificial Sequence
  Synthetic construct
acggacagcttacctttgga

SEQ ID NO: 28 - DNA, 20 bp, Artificial Sequence
  Synthetic construct
aagatggtgaaggtcggtgt

SEQ ID NO: 29 - DNA, 20 bp, Artificial Sequence
  Synthetic construct
tcctccctggagaagagcta

SEQ ID NO: 30 - DNA, 20 bp, Artificial Sequence
  Synthetic construct
agggacaggtcttggtgttg

SEQ ID NO: 31 - DNA, 20 bp, Artificial Sequence
  Synthetic construct
tgcttgtctggaacaactgc

SEQ ID NO: 32 - DNA, 24 bp, Artificial Sequence
  Synthetic construct
atttcactggcgagctcaggtact

SEQ ID NO: 33 - DNA, 24 bp, Artificial Sequence
  Synthetic construct
caccttcacacagagctgcagaaa

SEQ ID NO: 34 - DNA, 24 bp, Artificial Sequence
  Synthetic construct
agtgcctctttgctgctttcacac

SEQ ID NO: 35 - DNA, 24 bp, Artificial Sequence
  Synthetic construct
acatccatctttgtgccctaccct

SEQ ID NO: 36 - DNA, 24 bp, Artificial Sequence
  Synthetic construct
tcgcttcttgctaggactgaacca

SEQ ID NO: 37 - DNA, 20 bp, Artificial Sequence
  Synthetic construct
ttcatgaaccatcctctcca

SEQ ID NO: 38 - DNA, 22 bp, Artificial Sequence
  Synthetic construct
atgaggtccaccaccctgttgc

SEQ ID NO: 39 - DNA, 19 bp, Artificial Sequence
  Synthetic construct
gccggagccgttgtcgacg

SEQ ID NO: 40 - DNA, 20 bp, Artificial Sequence
  Synthetic construct
gcttggtggtttgctacgac

SEQ ID NO: 41 - DNA, 20 bp, Artificial Sequence
  Synthetic construct
aggacgggctcttcttcaaa

SEQ ID NO: 42 - DNA, 20 bp, Artificial Sequence
  Synthetic construct
aatgtccactgtgctgcttg

SEQ ID NO: 43 - DNA, 20 bp, Artificial Sequence
  Synthetic construct
gcttcccattctcagccttg

SEQ ID NO: 44 - DNA, 20 bp, Artificial Sequence
  Synthetic construct
acggatgtcaacgtcacact